(19) United States
(12) Patent Application Publication
(10) Pub. No.: US 2023/0346725 A1
(43) Pub. Date: Nov. 2, 2023

(54) IBUPROFEN-CONTAINING SOFT GELATIN CAPSULES

(71) Applicant: RECKITT BENCKISER HEALTH LIMITED, Berkshire (GB)

(72) Inventors: Georgia Kerry BROWN, Humberside (GB); Matthew Edward Anthony MCGIRR, Humberside (GB); Lucy Amber PHILLIPS, Humberside (GB)

(73) Assignee: RECKITT BENCKISER HEALTH LIMITED, Berkshire (GB)

(21) Appl. No.: 17/999,056

(22) PCT Filed: May 21, 2021
(86) PCT No.: PCT/GB2021/051240

§ 371 Date: Nov. 17, 2022

(30) Foreign Application Priority Data
May 21, 2020 (GB) 2007618.8

(51) Int. Cl. A61K 9/48; A61K 31/192

(52) U.S. Cl. CPC A61K 31/192; A61K 9/4825; A61K 9/485; A61K 9/4858; A61K 9/4866

(57) ABSTRACT

The present invention relates to a composition for encapsulation in a soft gelatin shell which comprises ibuprofen, one or more polyoxysorbitan esters and a base wherein the weight ratio of the one or more polyoxysorbitan esters to ibuprofen is from 0.0025:1 - 1.5:1, the weight ratio of the base to ibuprofen is from 1:3 - 1:12 and the weight ratio of the one or more polyoxysorbitan esters to base is 0.009:1 - 11: 1.

The present invention is directed to a composition that comprises ibuprofen. In particular, the present invention is directed to a composition that comprises ibuprofen and is suitable encapsulation in a soft gelatin capsule.

Soft gelatin capsules offer a number of advantages over tablets and caplets as a pharmaceutical dosage form. They are easily digested and dissolve quickly in the stomach thus allowing quicker onset of the desired pharmaceutical effect. Soft gelatin capsules are also very useful for administering poorly soluble or poorly absorbed active pharmaceutical ingredients (API). Soft gelatin capsules are also useful for protecting APIs from light and oxygen thus improving their stability.

Soft gelatin capsules which contain a pain killer, such as ibuprofen, are known in the art and are commercially available.

US 5 376 688 describes soft and hard gelatin capsules containing a fill formulation which comprises a diethylene glycol monoethyl ether and a polyglycerol oleate. US 5 912 011 discloses a solvent system for encapsulation in soft and hard gelatin capsules. WO 88/02625 discloses a solvent system to enhance the solubility of APIs. WO 2005/123133 discloses a solvent system for APIs which comprises 15 - 50 % by weight of polyethylene glycol.

However, while the soft gelatin capsules that are currently available exhibit a release rate and onset of pharmaceutical effect that is acceptable improvements in speed of action are continually sought after. The present invention provides a soft-gelatin capsule that works significantly quicker than current capsules.

According to an aspect of the present invention there is provided a composition for encapsulation in a soft gelatin shell which comprises ibuprofen, one or more polyoxysorbitan esters and a base wherein the weight ratio of the one or more polyoxysorbitan esters to ibuprofen is from 0.0025:1 - 1.5:1, the weight ratio of the base to ibuprofen is from 1:3 - 1:12 and the weight ratio of the one or more polyoxysorbitan esters to base is 0.009:1 - 11:1.

Preferably, the amount of ibuprofen in the composition is from 40 to less than 50% w/w.

Preferably the weight ratio of the one or more polyoxysorbitan esters to ibuprofen is 0.6:1-0.9:1. More preferably the weight ratio of the one or more polyoxysorbitan esters to ibuprofen is 0.7:1 - 0.9:1. Most preferably the weight ratio of the one or more polyoxysorbitan esters to ibuprofen is 0.8:1- 0.85:1.

Preferably the weight ratio of base to ibuprofen is 1:4 - 1:10. More preferably the weight ratio of base to ibuprofen is 1:5 - 1:7. Most preferably the weight ratio of base to ibuprofen is 1:5 - 1:6.

Preferably the weight ratio of the one or more polyoxysorbitan esters to base is 3:1 - 8.5:1. More preferably the weight ratio of the one or more polyoxysorbitan esters to base is 4.5:1-7.75:1.

Preferably the weight ratio of the one or more polyoxysorbitan esters to ibuprofen is 0.8:1-0.85:1, the weight ratio of base to ibuprofen is 1:5 - 1:6 and the weight ratio of the one or more polyoxysorbitan esters to base is 4.5:1 - 7.75:1.

The composition can comprise ibuprofen, one or more polyoxysorbitan esters and a base wherein the weight ratio of the ibuprofen: the one or more polyoxysorbitan esters:base is from about 15:15:1 to about 3:0.009:1.

Preferably the weight ratio of the ibuprofen: the one or more polyoxysorbitan esters:base is from about 10:8:1 to about 4:4:1.

The one or more polyoxysorbitan esters can be selected from the group consisting of polysorbate 20, polysorbate 21, polysorbate 40, polysorbate 60, polysorbate 61, polysorbate 65, polysorbate 80, polysorbate 81, polysorbate 85 or polysorbate 120. A preferred polyoxysorbitan ester is polysorbate 80.

The base can be selected from alkali metal hydroxides (i.e. the metals of Group I of the Periodic Table) particularly sodium and potassium, and alkali earth metal hydroxides (i.e. the metals of Group II of the Periodic Table) particularly calcium and magnesium. Preferred hydroxides are sodium hydroxide and potassium hydroxide. A more preferred hydroxide is potassium hydroxide.

The base can also be selected from carbonate and bicarbonate salts of the alkali and alkali earth metals, i.e. the metals of Group I or Group II of the periodic table.

The base can be selected from amines and amino acids such as ammonia, triethylamine, lysine or arginine.

The composition can further comprise polyethylene glycol in an amount from about 5% to about 20% w/w. Typically, the polyethylene glycol is a liquid at room temperature. The polyethylene glycol can have a number average molecular weight (Mn) of up to 1000. The polyethylene glycol can have a number average molecular weight (Mn) of from about 400 to about 800. Preferred polyethylene glycols can have a number average molecular weight (Mn) of 200, 300, 400, 600, 800. Preferred polyethylene glycols can have a number average molecular weight (Mn) of 400 or 600.

The composition can further comprise water in an amount from about 2% w/w to about 10% w/w. The water can be present at a level of from 3% w/w to 5.5% w/w.

Typically, the unit dose weight of the formulation is about 400 - 500 mg. More typically, the unit dose weight of the formulation is about 440 - 480 mg. Even more typically, the unit dose weight of the formulation is about 450 - 470 mg. Most typically, the unit dose weight of the formulation is about 460 mg.

Typically, the unit dose weight of the formulation is about 800 - 1000 mg. More typically, the unit dose weight of the formulation is about 850 - 950 mg. Even more typically, the unit dose weight of the formulation is about 900 - 940 mg. Most typically, the unit dose weight of the formulation is about 920 - 925 mg.

Typically, the unit dose has a fill volume of from 0.400 ml to 1.00 ml.

The composition can comprise an ibuprofen, one or more polyoxysorbitan esters, a hydroxide and a polyethylene glycol wherein ibuprofen is present at an amount of 40 - 50% w/w, the one or more polyoxysorbitan esters is present at an amount of 30 - 40% w/w, the hydroxide is present at an amount of 3 - 10% w/w, the polyethylene glycol is present at an amount of 5 - 20% w/w and the weight ratio of the one or more polyoxysorbitan esters to ibuprofen is 0.8:1 - 0.85:1, the weight ratio of base to ibuprofen is 1:5 - 1:6 and the weight ratio of the one or more polyoxysorbitan esters to base is 4.5:1 - 7.75:1.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a composition comprising:
(a) 35 - <50% w/w ibuprofen;
(b) 5 - 50% w/w One or more polyoxysorbitan esters; and
(c) 1 - 15% w/w A hydroxide.

An ibuprofen-containing composition comprising:
(a) 35 - <50% w/w ibuprofen;
(b) 5 - 50% w/w One or more polyoxysorbitan esters; and
(c) 1 - 15% w/w A hydroxide.
wherein the volume of the unit dose of the composition is from about 0.4 ml to about 1.0 ml.

The composition can comprise:
(a) 35 - <50% w/w ibuprofen;
(b) 30 - 50% w/w One or more polyoxysorbitan esters; and
(c) 2 - 15% w/w A hydroxide
wherein the volume of the unit dose of the composition is from about 0.4 ml to about 1 ml.

The composition can comprise:
(a) 35 - <50% w/w ibuprofen;
(b) 30 - 40% w/w One or more polyoxysorbitan esters;
(c) 2 - 15% w/w A hydroxide; and
(d) 5 - 15% w/w Polyethylene glycol
wherein the volume of the unit dose of the composition is from about 0.4 ml to about 1 ml.

The composition can consist essentially of:
(a) 35 - <50% w/w ibuprofen;
(b) 25 - 50% w/w One or more polyoxysorbitan esters;
(c) 2 - 15% w/w A hydroxide; and
(d) 1 - 10% w/w Water.

The composition can consist essentially of:
(a) 35 - 45% w/w ibuprofen;
(b) 35 - 50% w/w One or more polyoxysorbitan esters;
(c) 5 - 10% w/w A hydroxide; and
(d) 3 - 8% w/w Water.

The composition can consist essentially of:
(a) 40 - 45% w/w ibuprofen;
(b) 35 - 50% w/w One or more polyoxysorbitan esters;
(c) 4 - 12% w/w A hydroxide; and
(d) 1 - 10% w/w Water.

The composition can consist essentially of:
(a) 35 - <50% w/w ibuprofen;
(b) 25 - 50% w/w One or more polyoxysorbitan esters;
(c) 2 - 15% w/w A hydroxide; and
(d) 1 - 10% Water
wherein the volume of the unit dose of the composition is from about 0.4 ml to about 1 ml.

The composition can consist essentially of:
(a) 35 - 45% w/w ibuprofen;
(b) 35 - 50% w/w One or more polyoxysorbitan esters;
(c) 5 - 10% w/w A hydroxide; and
(d) 3 - 8% w/w Water
wherein the unit dose of the composition is from about about 0.4 ml to about 1 ml.

The composition can consist essentially of:
(a) 40 - 45% w/w ibuprofen;
(b) 35 - 50% w/w One or more polyoxysorbitan esters;
(c) 4 - 12% w/w A hydroxide; and
(d) 1 - 10% w/w Water
wherein the unit dose of the composition is from about 0.4 ml to about 1 ml.

The composition can comprise:
(a) 35 - <50% w/w ibuprofen;
(b) 30 - 40% w/w One or more polyoxysorbitan esters;
(c) 2 - 15% w/w A hydroxide; and
(d) 5 - 15% w/w Polyethylene glycol

The composition can consist essentially of:
(a) 35 - <50% w/w Ibuprofen;
(b) 0.1 - 50% w/w One or more polyoxysorbitan esters;
(c) 2 - 15% w/w A hydroxide;
(d) 0.1 - 40% w/w Polyethylene glycol; and
(e) 1 - 10% w/w Water.

The composition can consist essentially of:
(a) 35 - <50% w/w Ibuprofen;
(b) 10 - 50% w/w One or more polyoxysorbitan esters;
(c) 2 - 15% w/w A hydroxide;
(d) 10 - 40% w/w Polyethylene glycol; and
(e) 1 - 10% w/w Water.

The composition can consist essentially of:
(a) 40 - 45% w/w Ibuprofen;
(b) 35 - 40% w/w One or more polyoxysorbitan esters;
(c) 4 - 10% w/w A hydroxide;
(d) 5 - 15% w/w Polyethylene glycol; and
(e) 2 - 8% w/w Water.

The composition can consist essentially of:
(a) 40 - 45% w/w Ibuprofen;
(b) 35 - 40% w/w One or more polyoxysorbitan esters;
(c) 5 - 9% w/w A hydroxide;
(d) 5 - 10% w/w Polyethylene glycol; and
(e) 3 - 8% w/w Water.

The composition can comprise:
(a) 35 - <50% w/w ibuprofen;
(b) 30 - 40% w/w One or more polyoxysorbitan esters;
(c) 2 - 15% w/w A hydroxide; and
(d) 5 - 15% w/w Polyethylene glycol
wherein the volume of the unit dose of the composition is from about 0.4 ml to about 1 ml.

The composition can consist essentially of:
(a) 35 - <50% w/w Ibuprofen;
(b) 0.1 - 50% w/w One or more polyoxysorbitan esters;
(c) 2 - 15% w/w A hydroxide;
(d) 0.1 - 40% w/w Polyethylene glycol; and
(e) 1 - 10% w/w Water
wherein the unit dose of the composition is from about 0.4 ml to about 1 ml.

The composition can consist essentially of:
(a) 35 - <50% w/w Ibuprofen;
(b) 10 - 50% w/w One or more polyoxysorbitan esters;
(c) 2 - 15% w/w A hydroxide;
(d) 10 - 40% w/w Polyethylene glycol; and
(e) 1 - 10% w/w Water
wherein the unit dose of the composition is from about 0.4 ml to about 1 ml.

The composition can consist essentially of:
(a) 40 - 45% w/w Ibuprofen;
(b) 35 - 40% w/w One or more polyoxysorbitan esters;
(c) 4 - 10% w/w A hydroxide;
(d) 5 - 15% w/w Polyethylene glycol; and
(e) 2 - 8% w/w Water
wherein the unit dose of the composition is from about 0.4ml to about 1ml.

The composition can consist essentially of:
(a) 40 - 45% w/w Ibuprofen;
(b) 35 - 40% w/w One or more polyoxysorbitan esters;
(c) 4 - 9% w/w A hydroxide;
(d) 5 - 14% w/w Polyethylene glycol; and
(e) 3 - 8% w/w Water
wherein the unit dose of the composition is from about 0.4 ml to about 1 ml.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a soft gelatin capsule which contains a composition as described in any of the previous aspects.

According to an aspect of the present invention there is provided a soft gelatin capsule comprising a composition of ibuprofen wherein the composition allows for at least 5% of the ibuprofen dose to be solubilised within 5 minutes of immersion of the soft gelatine capsule in simulated gastric fluid at a pH of 1.2 .

Preferably the composition allows for at least 10% of the ibuprofen dose to be solubilised within 10 minutes of immersion of the soft gelatine capsule in simulated gastric fluid at a pH of 1.2. More preferably the composition allows for at least 10% of the ibuprofen dose to be solubilised within 5 minutes of immersion of the soft gelatine capsule in simulated gastric fluid at a pH of 1.2.

Preferably the composition allows for 15% of the ibuprofen dose to be solubilised within 10 minutes of immersion of the soft gelatine capsule in simulated gastric fluid at a pH of 1.2. More preferably the composition allows for at least 15% of the ibuprofen dose to be solubalised within 5 minutes of immersion of the soft gelatine capsule in simulated gastric fluid at a pH of 1.2.

Preferably the composition allows for at least 20% of the ibuprofen dose to be solubilised within 10 minutes of immersion of the soft gelatine capsile in simulated gastric fluid at a pH of 1.2. More preferably the composition allows for at least 20% of the ibuprofen dose to be solubilised within 5 minutes of immersion of the soft gelatine capsule in simulated gastric fluid at a pH of 1.2.

Preferably the composition allows for at least 30% of the ibuprofen dose to be solubilised within 10 minutes of immersion of the soft gelatine capsule in simulated gastric fluid at a pH of 1.2. More preferably the composition allow for at least 30% of the ibuprofen dose to be solubilised within 5 minutes of immersion of the soft gelatine capsule in simulated gastric fluid at a pH of 1.2.

Preferably the composition allows for at least 50% of the ibuprofen dose to be solubilised within 10 minutes of immersion of the soft gelatine capsule in simulated gastric fluid at a pH of 1.2. More preferably the composition allows for at least 50% of the ibuprofen dose to be solubilised within 5 minutes of immersion of the soft gelatine capsule in simulated gastric fluid at a pH of 1.2.

According to an aspect of the present invention there is provided a composition for a soft-gel capsule which comprises ibuprofen and a solvent system wherein the solvent system comprises:
a) from about 10 to about 30% w/w polyethylene glycol;
b) from about 40 to about 80% w/w of one or more polyoxysorbitan esters;
c) from about 5 to about 15% w/w of potassium hydroxide; and
d) water.

Typically, the solvent system comprises:
a) from about 10 to about 25% w/w polyethylene glycol;
b) from about 50 to about 70% w/w of one or more polyoxysorbitan esters;
c) from about 5 to about 15% w/w of potassium hydroxide; and
d) water.

Preferably, the solvent system comprises:
a) from about 10 to about 20% w/w polyethylene glycol;
b) from about 60 to about 65% w/w of one or more polyoxysorbitan esters;
c) from about 10 to about 15% w/w of potassium hydroxide; and
d) water.

Alternatively, the solvent system preferably comprises:
a) from about 20 to about 25% w/w polyethylene glycol;
b) from about 60 to about 65% w/w of one or more polyoxysorbitan esters;
c) from about 5 to about 10% w/w of potassium hydroxide; and
d) water.

Typically, the unit dose weight of the formulation is about 400 - 500 mg. More typically, the unit dose weight of the formulation is about 440 - 480 mg. Even more typically, the unit dose weight of the formulation is about 450 - 470 mg. Most typically, the unit dose weight of the formulation is about 460 mg.

Typically, the unit dose weight of the formulation is about 800 - 1000 mg. More typically, the unit dose weight of the formulation is about 850 - 950 mg. Even more typically, the unit dose weight of the formulation is about 900 - 940 mg. Most typically, the unit dose weight of the formulation is about 920 - 925 mg.

Typically, the composition has a release rate for ibuprofen of at least 5% in 5 mins. More typically, the composition has a release rate of at least 10% in 5 mins. Most typically, the composition has a release rate of at least 20% in 5 mins.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a soft-gel capsule which contains a composition which can comprise ibuprofen and a solvent system wherein the solvent system comprises:
a) from about 20 to about 25% w/w polyethylene glycol;
b) from about 60 to about 65% w/w of one or more polyoxysorbitan esters;
c) from about 5 to about 10% w/w of potassium hydroxide; and
d) water
wherein the composition has a weight of 440 - 480 mg and wherein the composition has a release rate for ibuprofen of at least 5% in 5 mins.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a soft-gel capsule which contains a composition which can comprise:
(a) 35 - <50% w/w ibuprofen;
(b) 30 - 40% w/w of one or more polyoxysorbitan esters;
(c) 2 - 15% w/w A hydroxide; and
(d) 5 - 15% w/w Polyethylene glycol
wherein the composition has a weight of 400 - 500 mg and wherein the composition has a release rate for ibuprofen of at least 5% in 5mins.

Typically, the unit dose weight of the formulation is about 440 - 480 mg. More typically, the unit dose weight of the formulation is about 450 - 470 mg. Most typically, the unit dose weight of the formulation is about 460 mg.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

According to an aspect of the present invention there is provided a soft-gel capsule which contains a composition which can comprise:
(a) 35 - <50% w/w ibuprofen;
(b) 30 - 40% w/w of one or more polyoxysorbitan esters;
(c) 2 - 15% w/w A hydroxide; and
(d) 5 - 15% w/w Polyethylene glycol
wherein the composition has a weight of 800 - 1000 mg and wherein the composition has a release rate for ibuprofen of at least 5% in 5mins.

Typically, the unit dose weight of the formulation is about 850 - 950 mg. More typically, the unit dose weight of the formulation is about 900 - 940 mg. Most typically, the unit dose weight of the formulation is about 920 - 925 mg.

The polyoxysorbitan ester can be selected from polysorbate 20, polysorbate 21, polysorbate 40, polysorbate 60, polysorbate 61, polysorbate 65, polysorbate 80, polysorbate 81, polysorbate 85 or polysorbate 120. Preferably, the polyoxysorbitan ester is polysorbate 80.

The polyethylene glycol can be selected from polyethylene glycols having a number average molecular weight (Mn) of from about 400 to about 800. Preferably the polyethylene glycol can have a number average molecular weight (Mn) of 400 or 600 or a combination thereof.

The composition can comprise a poloxysorbitan selected from polysorbate 20, polysorbate 21, polysorbate 40, polysorbate 60, polysorbate 61, polysorbate 65, polysorbate 80, polysorbate 81, polysorbate 85 or polysorbate 120 and a polyethylene glycol having a number average molecular weight (Mn) of from about 400 to about 800. Preferably the composition can comprise polysorbate 80 and a polyethylene glycol can have a number average molecular weight (Mn) of 400 or 600 or a combination thereof.

As used herein the reference to the solubilisation of ibuprofen refers to the release and solubilisation of an amount of the ibuprofen dose in simulated gastric fluid. For example, when the composition allows for the release and solubilisation of 5% of the ibuprofen dose within 5 minutes this means that within 5 minutes of immersion in simulated gastric fluid 5% of the ibuprofen dose has been is in solution. For example, if the ibuprofen dose is 100 mg then 5 mg of the ibuprofen will have been both released from the capsule and solubilised. An additional amount may have been released but will not have been solubilised within the 5 minute period following immersion of the gelatin capsule.

As used herein the reference to weight ratios refers to the perctange weight of the components in the composition. For example, a composition having a 40% by weight of a first component and 40% by weight of a second component has a weight ratio of 1:1.

As used herein the reference to the unit dose weight of the formulation refers to weight of the composition that would be filled into the gelatine shell. The terms ‘formulation’ and ‘composition’ are used interchangeably and have the same meaning.

For the avoidance of doubt, in accordance with the present invention it is the compositions which are for encapsulation within a gelatin shell that comprise the ibuprofen, base, polyoxysorbitan ester or polyethylene glycol and not the gelatin shell itself.

For the avoidance of doubt, the compositions described in the present specification are in the form of a liquid.

Embodiments of the invention will now be described by way of example only with reference to the accompanying Figures in which:

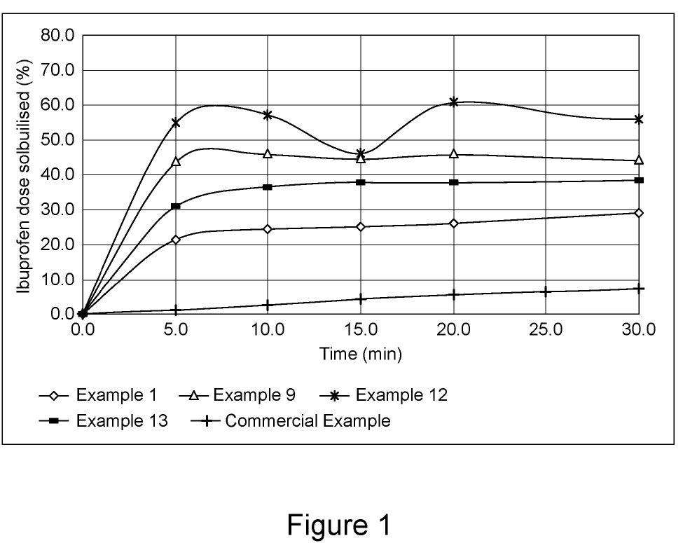

FIG. 1 illustrates release profiles for ibuprofen from compositions of the present invention and current commercially available soft gelatin capsules.

Tables 1- 4 illustrate examples of the composition of the present invention.

TABLE 1

| Component | Example 1 (% w/w) | Example 2 (% w/w) |
|---|---|---|
| Ibuprofen | 43.38 | 43.38 |
| Polysorbate 80 | 36.01 | 36.01 |
| PEG 400 | 12.74 | 7.59 |
| KOH Pellets | 4.72 | 7.81 |
| Water | 3.15 | 5.21 |
| Total | 100.00 | 100.00 |

TABLE 2

| Component | Example 3 (%w/w) | Example 4 (%w/w) | Example 5 (%w/w) | Example 6 (%w/w) |
|---|---|---|---|---|
| Ibuprofen | 43.38 | 43.38 | 43.38 | 43.38 |
| Polysorbate 80 | 48.64 | 36.82 | 11.93 | 0.11 |
| PEG 400 | 0.11 | 0.11 | 36.82 | 36.82 |
| KOH pellets | 4.72 | 11.81 | 4.72 | 11.81 |
| Purified water | 3.15 | 7.88 | 3.15 | 7.88 |
| Total | 100.00 | 100.00 | 100.00 | 100.00 |

TABLE 3

| Component | Example 7 (% w/w) | Example 8 (% w/w) | Example 9 (% w/w) | Example 10 (% w/w) |
|---|---|---|---|---|
| Ibuprofen | 43.38 | 43.38 | 43.38 | 43.38 |
| Polysorbate 80 | 36.01 | 36.01 | 36.01 | 36.01 |
| PEG 400 | 12.74 | 11.35 | 8.68 | 7.60 |
| KOH pellets | 4.72 | 5.55 | 7.16 | 7.81 |
| Purified water | 3.15 | 3.71 | 4.77 | 5.20 |
| Total | 100.00 | 100.00 | 100.00 | 100.00 |

TABLE 4

| Component | Example 11 (% w/w) | Example 12 (% w/w) | Example 13 (%w/w) |
|---|---|---|---|
| Ibuprofen | 43.38 | 43.38 | 48.21 |
| Polysorbate 80 | 48.75 | 38.22 | 40.01 |
| KOH pellets | 4.72 | 11.03 | 7.07 |
| Purified water | 3.15 | 7.37 | 4.71 |
| Total | 100.00 | 100.00 | 100.00 |

The formulation can be made using standard techniques known to the person of ordinary skill in the art. The polysorbate is initially heated to a temperature of 55° C. When polyethylene glycol is used the two materials are mixed together while heating to the same temperature. A portion of ibuprofen is added followed by a portion of aqueous potassium hydroxide solution while maintaining the heating. Once a homogenous solution has been achieved, the remaining ibuprofen and aqueous potassium hydroxide solution are added with continued heating. The resulting mixture is then stirred until a clear solution is obtained. The resulting solution can be stored until required for encapsulation in a gelatin capsule.

Similarly, the formulation can be encapsulated using standard soft gelatin encapsulation techniques well-known to the person skilled in the art. For example, the composition of the present invention can be deposited between two opposing ribbons of a gel composition. The composition of the ribbons may include gelatin and plasticisers such as glycerine and sorbitol and mixtures thereof. For example, a suitable gel composition can comprise about 35 - 50% by weight of gelatin, 20 - 40% by weight of a plasticiser and 25 - 50% by weight of water. The opposing ribbons are then run between two die rollers having die pockets thereon the surface of which corresponds to the configuration of the desired soft capsule. The composition is sealed within the fused casing.

The soft gelatin capsules were filled with compositions containing the standard dose of 200 mg of ibuprofen, i.e. in the examples above the amount of ibuprofen is 200 mg.

As can be seen from FIG. 1 and Table 5 below, the composition/pharmaceutical product of the present invention releases the ibuprofen unexpectedly and significantly quicker than a current commercial product tested under the same conditions.

TABLE 5

| Time | Example 1 | Example 9 | Example 12 | Example 13 | Commercial Example |
|---|---|---|---|---|---|
| 0.0 | 0.0 | 0.0 | 0.0 | 0.0 | 0.0 |
| 5.0 | 21.5 | 43.8 | 54.5 | 30.9 | 1.43 |
| 10.0 | 24.5 | 45.9 | 57.3 | 36.7 | 3.1 |
| 15.0 | 25.1 | 44.6 | 46.3 | 38.2 | 4.5 |
| 20.0 | 26.2 | 46.1 | 61.0 | 37.7 | 5.87 |
| 30.0 | 29.4 | 44.4 | 56.4 | 38.8 | 7.6 |

The release rate of the compositions of the present invention were determined by immersing the compositions in a gelatin capsule in simulated gastic fluid. The compositions were immersed into a beaker/container containing 900ml of the simulatied gastric fluid at a temperature of 37° C. The pH of the simulated gastric fluid was 1.2.

An advantage of the present invention is that there is provided a stable ibuprofen-containing formulation which is suitable for encapsulation in a soft gelatin capsule and which releases the ibuprofen in a more solubilisable form than standard ibuprofen-containing soft gelatin capsules. A further advantage is provided with the ability to use a lower amount of base within the formulation whilst improving the solubalisation of the ibuprofen in simulated gastric fluid.

Further modifications can be made without departing from the scope of the invention described herein.

1-120. (canceled)

121. A composition for encapsulation in a soft gelatin shell, the composition comprising:
ibuprofen;
one or more polyoxysorbitan esters; and
a base,
wherein a weight ratio of the one or more polyoxysorbitan esters to the ibuprofen is from 0.0025:1 - 1.5:1, a weight ratio of the base to the ibuprofen is from 1:3 - 1:12, and a weight ratio of the one or more polyoxysorbitan esters to the base is 0.009:1 - 11:1.

122. The composition as claimed in claim 121, wherein the amount of the ibuprofen in the composition is from 35 to less than 50% w/w.

123. The composition as claimed in claim 121, wherein the weight ratio of the one or more polyoxysorbitan esters to the ibuprofen is 0.6:1 - 0.9:1.

124. The composition as claimed in claim 123, wherein the weight ratio of the one or more polyoxysorbitan esters to the ibuprofen is 0.7:1 - 0.9:1.

125. The composition as claimed in claim 123, wherein the weight ratio of the one or more polyoxysorbitan esters to the ibuprofen is 0.8:1 - 0.85:1.

126. The composition as claimed in claim 121, wherein the weight ratio of the base to the ibuprofen is 1:4 - 1:10.

127. The composition as claimed in claim 126, wherein the weight ratio of the base to the ibuprofen is 1:5 - 1:7.

128. The composition as claimed in claim 126, wherein the weight ratio of the base to the ibuprofen is 1:5 - 1:6.

129. The composition as claimed in claim 121, wherein the weight ratio of the one or more polyoxysorbitan esters to the base is 3:1 - 8.5:1.

130. The composition as claimed in claim 129, wherein the weight ratio of the one or more polyoxysorbitan esters to the base is 4.5:1 - 7.75:1.

131. The composition as claimed in claim 121, wherein the weight ratio of the one or more polyoxysorbitan esters to the ibuprofen is 0.8:1 - 0.85:1, the weight ratio of the base to the ibuprofen is 1:5 - 1:6, and the weight ratio of the one or more polyoxysorbitan esters to the base is 4.5:1 - 7.75:1.

132. The composition as claimed in claim 121, wherein the composition comprises ibuprofen, one or more polyoxysorbitan esters, and a base,
wherein the weight ratio of the ibuprofen to the one or more polyoxysorbitan esters to the base is from about 15:15:1 to about 3:0.009:1.

133. The composition as claimed in claim 132, wherein the weight ratio of the ibuprofen to the one or more polyoxysorbitan esters to the base is from about 10:8:1 to about 4:4:1.

134. The composition as claimed in claim 121, wherein the composition further comprises water in an amount from about 2% w/w to about 10% w/w.

135. The composition as claimed in claim 134, wherein the water is present in an amount of from 3% w/w to 5.5% w/w.

136. The composition as claimed in claim 121, wherein the composition comprises:
40 - 50% w/w of ibuprofen;
30 - 40% w/w of one or more polyoxysorbitan esters;
3 - 10% w/w of a hydroxide; and
5 - 20% w/w of a polyethylene glycol,
wherein the weight ratio of the one or more polyoxysorbitan esters to the ibuprofen is 0.8:1 -0.85:1, the weight ratio of the base to the ibuprofen is 1:5 - 1:6, and the weight ratio of the one or more polyoxysorbitan esters to the base is 4.5:1 - 7.75:1.

137. A composition comprising:
35 - less than 50% w/w of ibuprofen;
5 - 50% w/w of one or more polyoxysorbitan esters; and
1 - 15% w/w of a hydroxide.

138. The composition as claimed in claim 137, wherein the composition consists essentially of:
35 - less than 50% w/w of the ibuprofen;
25 - 50% w/w of the one or more polyoxysorbitan esters;
2 - 15% w/w of the hydroxide; and
1 - 10% w/w of water.

139. The composition as claimed in claim 138, wherein the composition consists essentially of:
35 - 45% w/w of the ibuprofen;
35 - 50% w/w of the one or more polyoxysorbitan esters;
5 - 10% w/w of the hydroxide; and
3 - 8% w/w of water.

140. The composition as claimed in claim 138, wherein the composition consists essentially of:
40 - 45% w/w of the ibuprofen;
35 - 50% w/w of the one or more polyoxysorbitan esters;
4 - 12% w/w of the hydroxide; and
1 - 10% w/w of water.

141. The composition as claimed in claim 121, wherein the composition has a release rate for ibuprofen of at least 5% in 5 minutes.

142. A soft gelatin capsule which contains a composition as claimed in claim 121.

143. A soft gelatin capsule comprising a composition of ibuprofen, wherein the composition allows for at least 5% of the ibuprofen to be solubilized within 5 minutes of immersion of the soft gelatin capsule in simulated gastric fluid at a pH of 1.2.

144. The composition as claimed in claim 121, wherein the one or more polyoxysorbitan esters is selected from the group consisting of polysorbate 20, polysorbate 21, polysorbate 40, polysorbate 60, polysorbate 61, polysorbate 65, polysorbate 80, polysorbate 81, polysorbate 85 and polysorbate 120.
